(19) United States
(12) Patent Application Publication
(10) Pub. No.: US 2024/0192777 A1
(43) Pub. Date: Jun. 13, 2024

(54) METHOD, SYSTEM, AND ELECTRODE STRUCTURE FOR ACQUIRING ELECTRICAL SIGNALS FROM PLURALITY OF LEAD WIRES, AND METHOD FOR MANUFACTURING SAID ELECTRODE STRUCTURE

(71) Applicant: Meltin MMI Co., Ltd., Tokyo (JP)

(72) Inventors: Masahiro KASUYA, Tokyo (JP); Tatsuya SEKI, Tokyo (JP)

(73) Assignee: Meltin MMI Co., Ltd., Tokyo (JP)

(21) Appl. No.: 18/556,514

(22) PCT Filed: Mar. 31, 2022
(86) PCT No.: PCT/JP2022/016906

§ 371 Date: Oct. 20, 2023

(30) Foreign Application Priority Data
Apr. 21, 2021 (JP) 2021-071933

(51) Int. Cl. G06F 3/01; G05B 19/4155

(52) U.S. Cl. CPC G06F 3/015; G05B 19/4155; G05B 2219/36133

(57) ABSTRACT

This method for acquiring electrical signals from a plurality of lead wires uses an electrode structure to detect a plurality electrical signals from the plurality of lead wires, and includes: the feature wherein the electrode structure comprises a plurality of electrode layers arranged so as to be separated from each other, each of the plurality of electrode layers has a plurality of holes having dimensions such that the plurality of lead wires can pass therethrough, and each of the plurality of holes is connected to a conductor that conducts an electrical signal propagating through the lead wire passing through the hole to the outside of the electrode structure; and the feature of distinguishing between the detected plurality of electrical signals.

### TECHNICAL FIELD

The present disclosure relates to a method, system, and electrode structure for obtaining electrical signals from a plurality of lead wires, and a method of manufacturing said electrode structure. More specifically, the present disclosure relates to a method, etc. for obtaining electrical signals from a plurality of nerves of an organism.

### BACKGROUND ART

Development of a technology known as brain-machine interface (BMI) is ongoing. BMI refers to a technology for directly connecting the brain of an organism with a machine.

Information directly obtained from the brain through BMI can be utilized in various applications. For example, an object is moved based on information directly obtained from the brain or information obtained from an object is inputted into the brain, whereby the object is moved as if the object is the organism's own body, and a new sensory organ can be obtained. The object can be an external instrument, the organism's own body, another organism's body, etc. An external instrument may be, for example, a body assisting apparatus such as a prosthetic arm or a prosthetic leg, or a remotely controllable robot. Another organism's body is, for example, another organism's brain, which can be connected to a similar BMI.

### CITATION LIST

### Patent Literature

[PTL 1] International Publication No. WO 2018/147407

### SUMMARY OF INVENTION

### Technical Problem

The objective of the present invention is to provide methods, etc. for obtaining a plurality of electrical signals that propagate through a plurality of lead wires in a distinguishable form. More specifically, the objective of the present invention is to provide methods, etc. for obtaining a plurality of electrical signals that propagate through a plurality of nerves of an organism in a distinguishable form.

### Solution to Problem

To solve the aforementioned problem, the present invention provides, for example, the following items.

### (Item 1)

A method for obtaining electrical signals from a plurality of lead wires, comprising:
detecting a plurality of electrical signals from the plurality of lead wires by using an electrode structure, the electrode structure comprising a plurality of electrode layers that are disposed apart from one another, each of the plurality of electrode layers having a plurality of pores having a dimension that allows passage of the plurality of lead wires, and each of the plurality of pores being connected to a conductor for conducting an electrical signal that propagates through a passing lead wire out from the electrode structure; and
distinguishing the plurality of detected electrical signals.

### (Item 2)

The method of item 1, wherein the distinguishing comprises distinguishing the plurality of electrical signals based on a propagation rate of an electrical signal, a distance between the plurality of electrode layers, and a position of a pore and a time at which an electrical signal has been detected.

### (Item 3)

The method of item 1 or 2, wherein the plurality of pores have a first pore having a first dimension, and a second pore having a second dimension, which is different from the first dimension.

### (Item 4)

The method of any one of items 1 to 3, wherein
the plurality of electrode layers comprise at least a first electrode layer and a second electrode layer disposed adjacent to the first electrode layer, and
a central axis of at least one of the plurality of pores in the first electrode layer is offset from a central axis of at least one of the plurality of pores in the second electrode layer.

### (Item 5)

The method of item 4, wherein
the plurality of electrode layers comprise at least a first electrode layer and a second electrode layer disposed adjacent to the first electrode layer, and
a central axis of each of the plurality of pores in the first electrode layer is offset from a central axis of each of the plurality of pores in the second electrode layer.

### (Item 6)

The method of item 4 or 5, wherein the plurality of electrode layers further comprise a third electrode layer disposed adjacent to the second electrode layer, the second electrode layer is disposed between the first electrode layer and the third electrode layer, and the third electrode layer has substantially the same pore arrangement as the first electrode layer.

### (Item 7)

The method of item 6, wherein the plurality of electrode layers further comprise a fourth electrode layer disposed adjacent to the third electrode layer, the third electrode layer is disposed between the second electrode layer and the fourth electrode layer, and the fourth electrode layer has substantially the same pore arrangement as the second electrode layer.

### (Item 8)

The method of any one of items 1 to 7, wherein the number of the plurality of electrode layers is 3 or greater, or the number of the plurality of pores is 7 or greater.

### (Item 9)

The method of any one of items 1 to 8, wherein the plurality of lead wires are a plurality of nerves of an organism.

### (Item 10)

An electrode structure for obtaining electrical signals from a plurality of lead wires, the electrode structure comprising a plurality of electrode layers that are disposed apart from one another, each of the plurality of electrode layers having a plurality of pores having a dimension that allows passage of the plurality of lead wires, and each of the plurality of pores being connected to a conductor for conducting an electrical signal that propagates through a passing lead wire out from the electrode structure.

### (Item 11)

A system for obtaining electrical signals from a plurality of lead wires, comprising:
the electrode structure of item 10;
receiving means for receiving a plurality of electrical signals from the electrode structure; and
distinguishing means for distinguishing the plurality of electrical signals.

### (Item 12)

The system of item 11, wherein the distinguishing means is configured to distinguish the plurality of electrical signals based on propagation rates of the electrical signals, a distance between the plurality of electrode layers, and positions of pores and times at which an electrical signal has been detected.

### (Item 13)

A method of manufacturing an electrode structure for obtaining electrical signals from a plurality of lead wires, comprising the steps of:
preparing a plurality of electrode layers, each of the plurality of electrode layers having a plurality of pores having a dimension that allows passage of the plurality of lead wires, and each of the plurality of pores being connected to a conductor for conducting an electrical signal that propagates through a passing lead wire out from the electrode structure; and
disposing the plurality of electrode layers apart from one another.

### (Item 14)

The method of manufacturing of item 13, wherein
the plurality of lead wires are a plurality of nerves of an organism, and
the step of preparing a plurality of electrode layers comprises a step of determining the number of the plurality of electrode layers and the number of the plurality of pores so that the number of trajectories the nerves can take during a process of growth of the nerves is greater than the number of the plurality of nerves.

### (Item A1)

A method for obtaining electrical signals from a plurality of nerves of an organism, the method being executed by a processing unit, the method comprising:
receiving, by the processing unit, a plurality of electrical signals detected from the plurality of nerves by using an electrode structure, the electrode structure comprising a plurality of electrode layers, the plurality of electrode layers being disposed apart from one another in a direction toward which the plurality of electrode layers are overlaid, each of the plurality of electrode layers having a plurality of pores having a dimension that allows passage of the plurality of nerves, and each of the plurality of pores being connected to a conductor for conducting an electrical signal that propagates through a passing nerve out from the electrode structure; and
distinguishing, by the processing unit, one of the plurality of detected electrical signals from another one of the plurality of detected electrical signals based on positions of pores, at which the plurality of electrical signals have been detected, among the plurality of pores from another one of the plurality of detected electrical signals.

### (Item A2)

The method of item A1, wherein the distinguishing comprises: calculating a time at which an electrical signal can reach each pore from a propagation rate of the electrical signal and a distance between the plurality of electrode layers; identifying nerves passing through respective pores at which the one and the another one of the plurality of electrical signals have been detected at a time matching the calculated time; and identifying that the one and the another one of the plurality of electrical signals are electrical signals obtained from the respective identified nerves to distinguish the one from the another one of the plurality of electrical signals.

### (Item A3)

The method of item A1 or A2, wherein the plurality of pores have a first pore having a first dimension, and a second pore having a second dimension, which is different from the first dimension.

### (Item A4)

The method of any one of items A1 to A3, wherein
the plurality of electrode layers comprise at least a first electrode layer and a second electrode layer disposed adjacent to the first electrode layer, and
a central axis of at least one of the plurality of pores in the first electrode layer is offset from a central axis of at least one of the plurality of pores in the second electrode layer.

### (Item A5)

The method of item A4, wherein
the plurality of electrode layers comprise at least a first electrode layer and a second electrode layer disposed adjacent to the first electrode layer, and
a central axis of each of the plurality of pores in the first electrode layer is offset from a central axis of each of the plurality of pores in the second electrode layer.

### (Item A6)

The method of item A4 or A5, wherein the plurality of electrode layers further comprise a third electrode layer disposed adjacent to the second electrode layer, the second electrode layer is disposed between the first electrode layer and the third electrode layer, and the third electrode layer has substantially the same pore arrangement as the first electrode layer.

### (Item A7)

The method of item A6, wherein the plurality of electrode layers further comprise a fourth electrode layer disposed adjacent to the third electrode layer, the third electrode layer is disposed between the second electrode layer and the fourth electrode layer, and the fourth electrode layer has substantially the same pore arrangement as the second electrode layer.

### (Item A8)

The method of items A1 to A7, wherein the number of the plurality of electrode layers is 3 or greater, or the number of the plurality of pores is 7 or greater.

### (Item A9)

An electrode structure for obtaining electrical signals from a plurality of nerves of an organism, the electrode structure comprising a plurality of electrode layers, the plurality of electrode layers being disposed apart from one another in a direction toward which the plurality of electrode layers are overlaid, each of the plurality of electrode layers having a plurality of pores having a dimension that allows passage of the plurality of nerves, and each of the plurality of pores being connected to a conductor for conducting an electrical signal that propagates through a passing nerve out from the electrode structure.

### (Item A10)

A system for obtaining electrical signals from a plurality of nerves of an organism, comprising:
the electrode structure of item A9;
receiving means for receiving a plurality of electrical signals from the electrode structure; and
distinguishing means for distinguishing one of the plurality of electrical signals from another one of the plurality of detected electrical signals based on positions of pores, at which the plurality of electrical signals have been detected, among the plurality of pores.

### (Item A11)

The system of item A10, wherein the distinguishing means is configured to calculate a time at which an electrical signal can reach each pore from propagation rates of the electrical signals and a distance between the plurality of electrode layers, identify nerves passing through respective pores at which the one of the plurality of electrical signals and the another one of the plurality of electrical signals have been detected at a time matching the calculated time, and identify that the one of the plurality of electrical signals and the another one of the plurality of electrical signals are electrical signals obtained from the respective identified nerves to distinguish the one of the plurality of electrical signals from the another one of the plurality of electrical signals.

### (Item A12)

The system of any of the preceding items for use in a brain-machine interface (BMI).

### (Item A13)

The system of item A12, wherein
the electrode structure is embedded into a body of a subject, and
the receiving means receives a plurality of electrical signals emitted from a brain of the subject via the electrode structure embedded into the body of the subject.

### (Item A14)

A method of manufacturing an electrode structure for obtaining electrical signals from a plurality of nerves of an organism, comprising the steps of:
preparing a plurality of electrode layers, each of the plurality of electrode layers having a plurality of pores having a dimension that allows passage of the plurality of nerves, and each of the plurality of pores being connected to a conductor for conducting an electrical signal that propagates through a passing nerve out from the electrode structure; and
disposing the plurality of electrode layers apart from one another in a direction toward which the plurality of electrode layers are overlaid.

### (Item A15)

The method of manufacturing of item A14, wherein the step of preparing a plurality of electrode layers comprises a step of determining the number of the plurality of electrode layers and the number of the plurality of pores so that the number of trajectories a nerve can take during a process of growth of the nerve is greater than the number of the plurality of nerves.

### Advantageous Effects of Invention

The present invention can obtain a plurality of electrical signals that propagate through a plurality of lead wires in a distinguishable form, whereby information can be extracted from the plurality of electrical signals that propagate through the plurality of lead wires. The present invention can be utilized in, for example, BMI, whereby a plurality of electrical signals that propagate through a plurality of nerves of an organism can be obtained in a distinguishable form.

### BRIEF DESCRIPTION OF DRAWINGS

FIG. 1 Diagram showing an example of electrode layer 110 according to one embodiment of the invention.

FIG. 2 Diagram showing an example of electrode structure 100 according to one embodiment of the invention.

FIG. 3 Perspective view an example of an arrangement of two electrode layers in electrode structure 100 according to one embodiment of the invention.

FIG. 4 Diagram showing a specific example of two electrode layers in electrode structure 100 according to one embodiment of the invention.

FIG. 5A Diagram showing an example of a plurality of nerves growing in electrode structure 100 according to one embodiment of the invention.

FIG. 5B Diagram showing an example of a plurality of nerves growing in electrode structure 100 according to one embodiment of the invention.

FIG. 5C Diagram showing an example of a plurality of nerves growing in electrode structure 100 according to one embodiment of the invention.

FIG. 5D Diagram showing an example of a plurality of nerves growing in electrode structure 100 according to one embodiment of the invention.

FIG. 6 Diagram showing an example of a configuration of system 1000 for obtaining electrical signals from a plurality of lead wires, according to one embodiment of the invention.

FIG. 7 Flow chart showing an example of procedure 700 for obtaining electrical signals from a plurality of lead wires.

FIG. 8 Flow chart showing an example of procedure 800 for manufacturing electrical structure 100 for obtaining electrical signals from a plurality of lead wires.

FIG. 9 Diagram schematically showing an example of system 1000 for obtaining electrical signals from a plurality of lead wires according to one embodiment of the invention when used in BMI.

### DESCRIPTION OF EMBODIMENTS

The present invention is described hereinafter. The terms used herein should be understood as being used in the meaning that is commonly used in the art, unless specifically noted otherwise. Therefore, unless defined otherwise, all terminologies and scientific technical terms that are used herein have the same meaning as the general understanding of those skilled in the art to which the present invention pertains. In case of a contradiction, the present specification (including the definitions) takes precedence.

### (Definition of the Terms)

As used herein, “lead wire” refers to a string-like element, which is capable of transmitting an electrical signal. In this regard, “string-like” refers to an elongated shape like a string. More specifically, this refers to a shape with the longitudinal dimension that is greater (e.g., about 5-fold or more, about 10-fold or more, about 50-fold or more, or about 100-fold or more) than the transverse dimension. A “lead wire” may be, for example, an electrically conductive member (e.g., copper wire, wire, cable, etc.) or a tissue that can transmit an electrical signal via a chemical ion (e.g., nerve of an organism, etc.).

As used herein, “organism” refers to a live organism. An organism is not limited to humans and includes, for example, animals, plants, etc.

As used herein, “about” refers to a numerical range of +10% from the numerical value that is described subsequent to “about”.

The embodiments of the invention are described hereinafter with reference to the drawings.

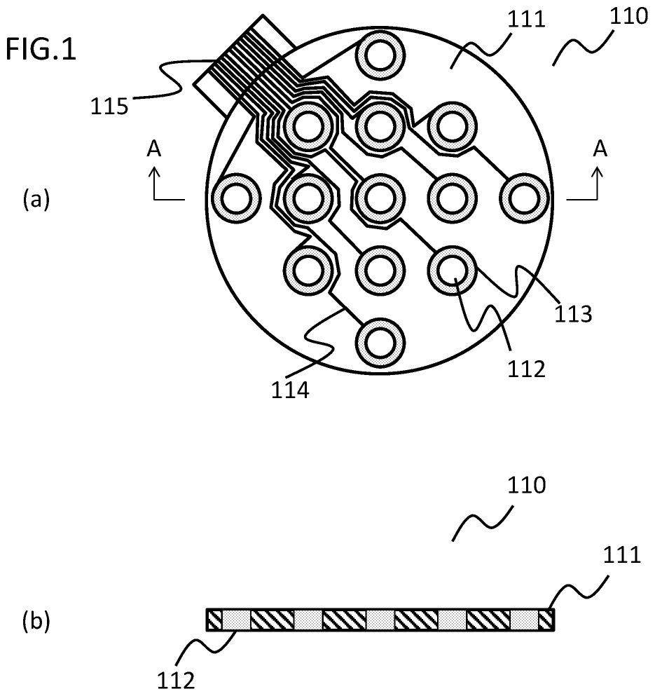

FIG. 1 shows an example of an electrode layer 110 according to one embodiment of the invention. FIG. 1 (a) shows a plane view of the electrode layer 110. FIG. 1 (b) shows an A-A cross-sectional view of the electrode layer 110 along line A-A in FIG. 1 (a).

The electrode layer 110 comprises a substrate 111, a plurality of pores 112, and conductors 113 and 114. The electrode layer 110 can further comprise an output terminal 115.

The substrate 111 is a member that is the base of the electrode layer 110. In the example shown in FIG. 1, the substrate 111 is illustrated as a substantially circular plate, but the shape of the substrate 111 is not limited thereto. The substrate 111 can have any planar shape. The substrate 111 can have a planar shape such as a substantially circular shape, substantially oval shape, substantially egg shape, substantially triangular shape, substantially quadrangular shape, or substantially polygonal shape.

The substrate 111 can have any dimension in plane view. The dimension of the substrate 111 can be a dimension in accordance with the application thereof. For example, the substrate 111 for obtaining an electrical signal from a nerve of an organism can have a dimension that fits within a square with a side of about 100,000 μm, a dimension that fits within a square with a side of about 50,000 μm, a dimension that fits within a square with a side of about 10,000 μm, a dimension that fits within a square with a side of about 5,000 μm, a dimension that fits within a square with about a side of 3,000 μm, etc. More specifically, the substrate 111, when the planar shape thereof is for example substantially circular, can have a diameter of about 10,000 μm or less, a diameter of about 5,000 μm or less, or a diameter of about 3,000 μm or less. Preferably, the substrate 111 can have a diameter of about 500 μm.

The substrate 111 can have any thickness. The thickness of the substrate 111 can be a thickness in accordance with the application thereof. For example, the substrate 111 for obtaining an electrical signal from a nerve of an organism can have a thickness of about 100 μm or less, a thickness of about 50 μm or less, a thickness of about 30 μm or less, a thickness of about 10 μm or less, etc. Preferably, the thickness of the substrate 111 can be about 20 μm.

The substrate 111 can be formed of any non-electrically-conductive material. Examples of materials for forming the substrate 111 include, but are not limited to, polyimide. Preferably, a material for forming the substrate 111 may be coated with a biocompatible material. The electrode layer 110 or an electrode structure comprised of the electrode layer 110 can be embedded into an organism by the substrate 111 being formed of a biocompatible material, and an electrical signal that propagates through a nerve of an organism can be obtained by using the electrode layer of the 110 or the electrode structure. Examples biocompatible material include, but are not limited to, MPC polymers.

A plurality of pores 112 are formed to penetrate through the substrate 111. In the example shown in FIG. 1, the plurality of pores 112 are illustrated as substantially circular pores, but the shape of the plurality of pores 112 are not limited thereto. The plurality of pores 112 can have any planar shape. The plurality of pores 112 can have a planar shape such as a substantially circular shape, substantially oval shape, substantially egg shape, substantially triangular shape, substantially quadrangular shape, or substantially polygonal shape. Each of the plurality of pores 112 may have, for example, the same shape from one another, or at least one of the plurality of pores 112 may have a different shape from other pores of the plurality of pores 112.

The plurality of pores 112 can have any dimension in a plane view. The dimension of the plurality of pores 112 is a dimension in accordance with the dimension of a target lead wire from which an electrical signal is obtained. Specifically, the dimension of the plurality of pores 112 can be a dimension that allows passage of a target lead wire from which an electrical signal is obtained, whereby the electrode layer 110 can obtain an electrical signal from the lead wire passing through the pore. For example, each of the plurality of pores 112 for obtaining an electrical signal from a nerve of an organism can have a dimension that fits within a square with a side of about 1,000 μm, a dimension that fits within a square with a side of about 500 μm, a dimension that fits within a square with a side of about 300 μm, a dimension that fits within a square with a side of about 120 μm, etc. More specifically, the plurality of pores 112, when the planar shape thereof is for example substantially circular, can have a diameter of about 1000 μm or less, a diameter of about 500 μm or less, a diameter of about 300 μm or less, or a diameter of about 120 μm or less. Preferably, the plurality of pores 112 can have a diameter of about 120 μm. For example, each of the plurality of pores 112 may have the same dimension from one another, or at least one of the plurality of pores 112 may have a dimension that is different from other pores of the plurality of pores 112. In a preferred embodiment, the plurality of pores 112 can have a first pore and a second pore having a dimension that is different from that of the first pore.

The number of the plurality of pores 112 is any number that is two or greater. The number of the plurality of pores 112 is, for example, 10 or greater. Preferably, the number of the plurality of pores 112 is determined in accordance with the number of the plurality of target lead wires from which an electrical signal is obtained. For example, the number of plurality of pores 112 for obtaining an electrical signal from a nerve of an organism can be determined with the number of the plurality of electrode layers 110 in the electrode structure 100 based on the number of trajectories a nerve can take during a process of growth of the nerve as discussed below.

The interval between the plurality of pores 112 can have any value. The interval between the plurality of pores 112 is an interval in accordance with the dimension of a target lead wire from which an electrical signal is obtained. For example, the interval between the plurality of pores 112 for obtaining an electrical signal from a nerve of an organism can be, between the two closest pores, about 10 μm or less, about 50 μm or less, about 100 μm or less, about 300 μm or less, about 600 μm or less, about 1,000 μm or less, about 1,200 μm or less, about 1,500 μm or less, etc. Preferably, the interval between two pores 112 can be about 600 μm or about 1200 μm.

The conductors 113 and 114 are configured to conduct an electrical signal that propagates through a lead wire passing through the plurality of pores 112 out from the electrode layer. A conductor includes a first conductor 113 disposed on the circumferential edge of the plurality of pores 112 and a second conductor 114 connected to the first conductor 113 and the output terminal 115.

The first conductor 113 can be disposed on not only the circumferential edge of the plurality of pores 112, but also the inner surface of the plurality of pores 112 (see FIG. 1 (b)), whereby the probability of a lead wire passing through a pore contacting the first conductor 113 can be increased. The first conductor 113 may be disposed on a part of the inner surface of a pore, or the entire inner surface of a pore. The conductor 113 can be an annular ring.

In the example shown in FIG. 1, the first conductor 113 is disposed on the substrate 111 in a substantially circular shape, but the first conductor 113 can be disposed on the substrate 111 in any shape. The first conductor 113 can be disposed on the substrate 111 in a shape such as a substantially circular shape, substantially oval shape, substantially egg shape, substantially shape, substantially quadrangular shape, or substantially polygonal shape.

The second conductor 114 is placed on the substrate 111 to connect the first conductor 113 with the output terminal 115. The second conductor 114 is placed in a manner that it is electrically insulated from other first conductors 113 and other second conductors 114. Specifically, an electrical signal from a lead wire passing through a pore can be conducted from the first conductor 113 of the pore to an output terminal without being mixed with an electrical signal from a lead wire passing through another pore, whereby an electrical signal retrieved from the output terminal 115 can be distinguished as to which pore that electrical signal has been obtained from.

The second conductor 114 connects to the first conductor 113 and the output terminal 115 through any route, as long as it is electrically insulated from other first conductors 113 and other second conductors 114.

The conductors 113 and 114 can be formed from any material that can conduct an electrical signal. Examples of materials with which the conductors 113 and 114 are formed limited include, but are not to, stainless steel. Preferably, the material with which the conductors 113 and 114 are formed is a biocompatible material. The conductors 113 and 114 being a biocompatible material can facilitate the electrode layer 110 or an electrode structure comprised of the electrode layer 110 from being embedded into an organism. Examples of the biocompatible material include, but are not limited to, platinum.

The output terminal 115 is configured so that an electrical signal can be outputted from the electrode layer 110. An electrical signal can be outputted out from the electrode layer 110 via the output terminal 115.

The output terminal 115 may be configured to not only output an electrical signal, but also receive an electrical signal from the outside and communicate the received electrical signal to a corresponding pore, whereby an electrical signal can be conducted to a lead wire passing through a pore (e.g., stimulate a nerve when the lead wire is a nerve).

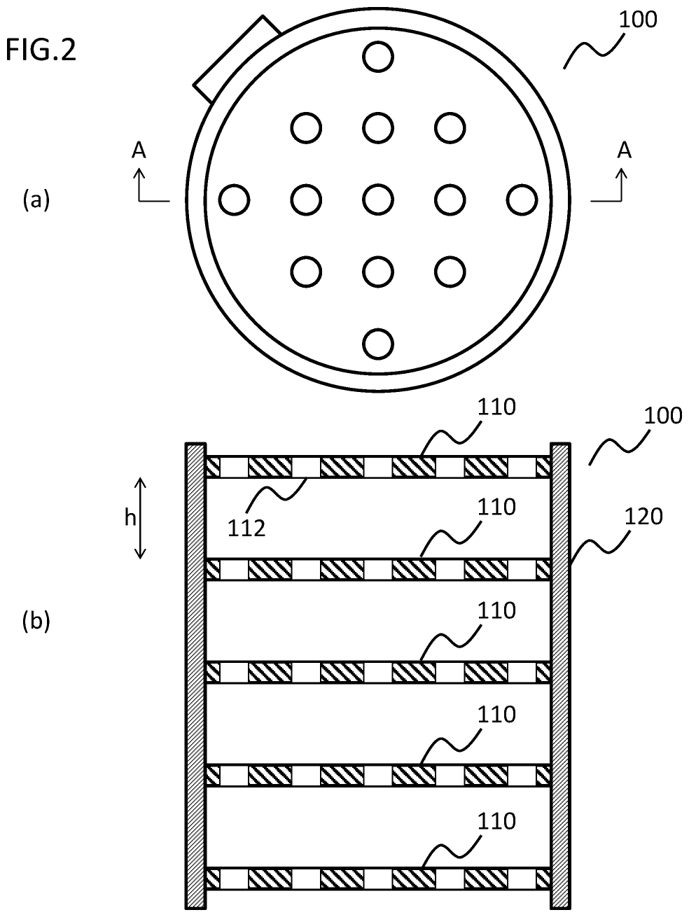

FIG. 2 shows an example of the electrode structure 100 according to one embodiment of the invention. FIG. 2 (a) shows a plane view of the electrode structure 100. FIG. 2 (b) shows an A-A cross-sectional view of the electrode structure 100 along line A-A in FIG. 2 (a).

In the example shown in FIG. 2, the conductors 113 and 114 that the electrode layer 110 can comprise are not shown to simplify the illustration. It is understood that each of the electrode layers 110, which the electrode structure 100 comprises, comprises the conductors 113 and 114 described with reference to FIG. 1.

The electrical structure 100 comprises a plurality of electrode layers 110.

In the example shown in FIG. 2, the electrode structure 100 comprises five electrode layers 110, but the electrode structure 100 can comprise any number of electrode layers 110, which is two or greater. For example, the number of electrode layers 110 is 5 or greater. Preferably, the number of a plurality of the electrode layers 110 is determined in accordance with the number of the plurality of target lead wires from which an electrical signal is obtained. As described below, it is preferable that each of the plurality of lead wires passes through an electrode structure through a route that is unique to each of the plurality of lead wires. Thus, the number of the plurality of the electrode layers 110 and the number of the plurality of pores 112 of each electrode layer 110 are preferably the number at which each of the plurality of lead wires can follow a unique route. For example, the number of the plurality of electrode layers 110 for obtaining an electrical signal from a nerve of an organism can be determined with the number of the plurality of pores 112 of each electrode layer 110 based on the number of trajectories a nerve can take during a process of growth of the nerve.

For example, the number m of the plurality of electrode layers 110 and the number n of the plurality of pores 112 of each electrode layer can be determined as follows.

Firstly, the number fk of pores that a nerve which has passed through a pore in the k-th layer can pass through in the next k+1-th layer is represented by

$${f}_{k} = {f}_{c ( ratio )} . , \qquad [ Numeral 1 ]$$

$$wherein$$

$$ratio = \frac{\frac{l}{m + 1} tan θ}{r} , \qquad [ Numeral 2 ]$$

$$r = {r}^{′} + {d}_{a} + {d}_{trace} . ,$$

wherein e is the maximum value of a trajectory that curves when a nerve grows, r′ is the radius of a pore, l is the length of the electrode structure 100, m is the number of layers, dₐ is the width of the conductor 113 (annular ring), d_{trace} is the minimum distance that the conductors 113 and 114 must be separated on the substrate, and f_{c(ratio)} is a function representing the number of circles that can be laid without overlapping when circles with a diameter of 1 (unit circles) are laid within a circle with a diameter=ratio.

Secondly, the number of cases after passing through all m layers is

n·f₂·fₛ . . . . . fₘ (l), [Numeral 3]

wherein the number of pores of the first electrode layer is n.

Thirdly, if f is assumed to have the same value in all layers to simplify the calculation, (l) can be represented by

n·f^{m-1} [Numeral 4]

The number of cases only needs to exceed the number N of a plurality of nerves of a subject, so that m and n can be selected to satisfy

$$f > {( \frac{α \cdot N}{n} )}^{\frac{1}{m - 1}} . , \qquad [ Numeral 5 ]$$

wherein a is the safety factor.

For example, calculation under the conditions of about 2800 rat sciatic nerves, safety factor of 3, and θ=60 can result in m=5 when n=7 is substituted in. If a value in the actually manufacturable range is searched to a certain extent in the vicinity thereof, (m, n)=(5,7), (3,21), etc. can be obtained.

The plurality of electrode layers 110 are disposed apart at an interval h in the electrode structure 100. The interval h can be any value. The interval h can be a value in accordance with the dimension of a target lead wire from which an electrical signal is obtained. The interval h can be, for example, a factor of the interval between a plurality of pores (e.g., about √2-fold, √3-fold, about 2-fold, about 3-fold, etc.). For example, for the electrode structure 100 for obtaining an electrical signal from a nerve of an organism, interval h can be about 500 μm or less, about 1,000 μm or less, about 1,500 μm or less, about 2,000 μm or less, etc. Preferably, interval h can be about 1,250 μm or less.

In the example shown in FIG. 2, the plurality of electrode layers 110 are connected to one another by a connection member 120 that the electrode structure 100 can comprise. In the electrode structure 100, the plurality of electrode layers 110 may be connected to one another with another member as shown in FIG. 2, or the plurality of electrode layers 110 may be integrated.

When an electrical signal is obtained from a plurality of lead wires by using the electrode structure 100, the plurality of lead wires are in a state where the wires have passed through the electrode structure 100. At this time, the plurality of lead wires pass through one pore of each electrode layer. Each of the plurality of lead wires preferably passes through the electrode structure via a route that is unique to each of the plurality of lead wires. Specifically, it is preferable that there are the same number of routes as the number of the plurality of lead wires. This is because when an electrical signal is obtained from a plurality of lead wires by using the electrode structure 100, each of the plurality of lead wires having a unique route enables identification of which of the plurality of lead wires the obtained electrical signal is obtained from. Specifically, identification of which conductor 114 among the plurality of conductors 114 of the output terminal 115 of each electrode layer the detected electrical signal is obtained from enables identification of which pore the electrical signal is detected from. The unique route, and in turn the lead wire corresponding to the route, can be identified.

For example, even when electrical signals propagate simultaneously through a plurality of lead wires, it is possible to identify which lead wire among the plurality of lead wires the electrical signal is obtained from. For example, a plurality of electrical signals or noises can be identified from propagation rates of electrical signals propagating through a lead wire, a distance between electrode layers, position of pores at which an electrical signal has been detected, and times at which an electrical signal has been detected because the time at which an electrical signal can reach each pore can be calculated from the propagation rate of the electrical signal and the distance between the electrode layers, and the pore at which the electrical signal has been detected at the time matching the calculated time can be identified as the pore through which the electrical signal has passed.

In the case, for example, an electrical signal is obtained from a plurality of nerves by using the electrode structure 100, the plurality of nerves can grow to pass through the electrode structure 100. For example, a plurality of nerves would pass through pores of each electrode layer of the electrode structure 100 by cutting existing nerves in an organism, inserting the electrode structure 100 between the cut nerves, and then allowing the cut nerves to grow. The inventors found that: if the pores are too small, the growth of the nerves is itself inhibited such that the nerves may not successfully grow; if there are too few pores, a route unique to each of the plurality of nerves may not be able to be secured; and if the pores are too large, it would be difficult to place a conductor on a substrate and/or the size of the substrate would be too large. The inventors found a pore configuration (e.g., size, number, and arrangement of pores), which can secure an area where a conductor can be disposed on a substrate while increasing the probability of a plurality of nerves to pass through a pore without inhibiting the growth of the plurality of nerves, by suitably setting the pore configuration.

When a plurality of nerves are passed through the electrode structure 100 by allowing cut nerves to grow, an electrical signal conducted through a nerve would have two routes, i.e., the original signal route and a signal route from the electrode structure 100 to the outside. If, for example, there is a hand or a foot that is inherently controlled beyond the original signal route, an electrical signal conducted through a nerve can move the hand or foot together with an external instrument. For example, it may not be possible to move only a hand of an external instrument (e.g., robot) without moving a user's hand. For example, a dynamic clamp, etc. may be used to block the action potential at the original signal path for the prevention thereof.

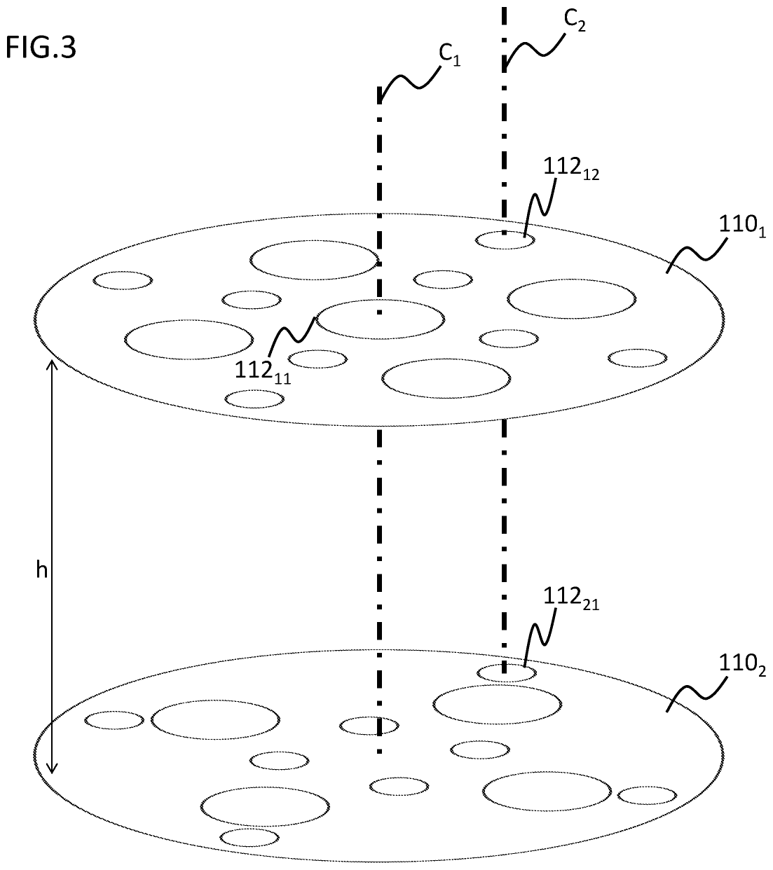

FIG. 3 is a perspective view showing an example of an arrangement of two electrode layers in the electrode structure 100 according to one embodiment of the invention. The two electrode layers may be a part or all of the electrode layers that the electrode structure 100 comprises.

In the example shown in FIG. 3, the conductors 113 and 114 and the output terminal 115 that a first electrode layer 110₁ and a second electrode layer 110₂ can comprise are not shown to simplify the illustration. It is understood that the first electrode layer 110₁ and second electrode layer 110₂ comprise the conductors 113 and 114 and the output terminal 115 described in reference to FIG. 1. In the example shown in FIG. 3, the connection member 120 for connecting the first electrode layer 110₁ and second electrode layer 110₂ is also not shown to simplify the illustration.

The first electrode layer 110₁ and the second electrode layer 110₂ are disposed apart at interval h. Each of the first electrode layer 110₁ and second electrode layer 110₂ has a first pore having a first diameter and a second pore having a diameter that is smaller than the first diameter.

The first electrode layer 110₁ and second electrode layer 110₂ can secure an area where the conductor 113 and/or conductor 114 can be disposed on the substrate 111 while increasing the probability of a plurality of lead wires to pass through a pore by having a plurality of pores with different diameters.

Furthermore, the first electrode layer 110₁ and the second electrode layer 110₂ are disposed so that a central axis of at least one of the plurality of pores in the first electrode layer 110₁ is offset from a central axis of at least one of the plurality of pores in the second electrode layer 110₂.

For example, a first pore 112₁₁ of the first electrode layer 110₁ has a first central axis C₁, and the first central axis C₁ does not match the central axis of any of the plurality of pores of the second electrode layer 110₂. Specifically, the first central axis C₁ is offset from the axis of each of the plurality of pores of the central second electrode layer 110₂. In view of a central axis of a pore of the first electrode layer 110₁ being offset from a central axis of a pore of the second electrode layer 110₂ in this manner, a lead wire passing through a pore of the first electrode layer 110₁ would diverge away from the direction of the central axis C₁ to pass through a pore of the second electrode layer 110₂. The direction of divergence can be random. Thus, this can lead to randomization of a route a lead wire can take and in turn a route unique to a lead wire.

Meanwhile, a second pore 112₁₂ of the first electrode layer 110₁ has a second central axis C₂. The second central axis C₂ matches the central axis of a first pore 112₂₁ of the second electrode layer 110₂.

The example shown in FIG. 3 describes that some of the pores of the first electrode layer 110₁ share a central axis with some of the pores of second electrode layer 110₂, but the present invention is not limited thereto. In a preferred embodiment, the first electrode layer 110₁ and the second electrode layer 110₂ may be disposed so that a central axis of each of the plurality of pores of the first electrode layer 110₁ is offset from a central axis of each of the plurality of pores of second electrode layer 110₂, whereby randomization of a route a plurality of lead wires can take can be promoted.

When the electrode layers shown in FIG. 3 form an electrode structure for obtaining electrical signals from a plurality of lead wires, electrode layers having substantially the same pore arrangement as the first electrode layer 110₁ and electrode layers having substantially the same pore arrangement as the second electrode layer 110₂ can be alternatingly disposed. For example, the second electrode layer 110₂ can be disposed adjacent to the first electrode layer 110₁ and a third electrode layer having substantially the same pore arrangement as the first electrode layer 110₁ can be disposed adjacent to the second electrode layer 110₂, whereby the second electrode layer would be disposed between the first electrode layer and the third electrode layer. Furthermore, a fourth electrode layer having substantially the same pore arrangement as the second electrode layer 110₂ can also be disposed adjacent to the third electrode layer, whereby the third electrode layer would be disposed between the second electrode layer and the fourth electrode layer. In any adjacent electrode layers in such an electrode structure, a central axis of at least one of a plurality of pores of one of the electrode layers would be offset from a central axis of at least one of the plurality of pores of the other electrode layer.

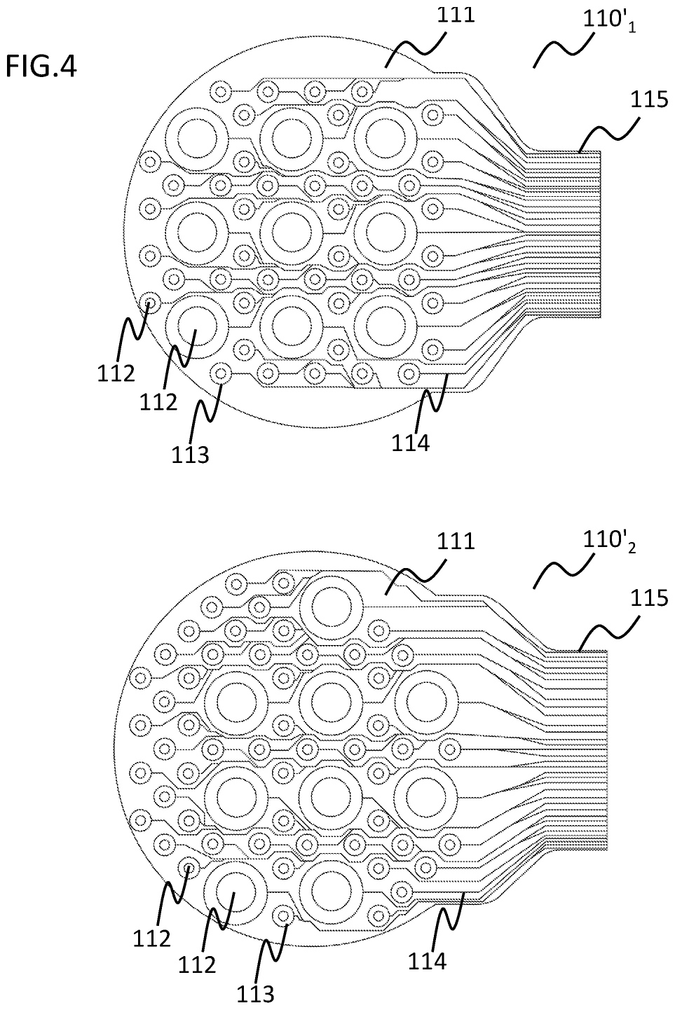

FIG. 4 shows a specific example of two electrode layers in the electrode structure 100 according to one embodiment of the invention.

A first electrode layer 110′₁ has a substantially circular substrate 111, and has a plurality of pores 112, a first conductor 113, a second conductor 114, and an output terminal 115 on the substrate 111.

In this example, the substrate 111 has a diameter of about 5,000 μm, and has an extension section for placing the second conductor 114 on the output terminal 115 side. The extension section has a dimension of about 980 μm.

The plurality of pores 112 have first pores having a first diameter and second pores having a smaller diameter than the first diameter. The first pores have a diameter of about 500 μm, and the first conduct 113 is disposed around the first pores at a diameter of about 800 μm. The interval between the first pores is about 1,200 μm. The second pores have a diameter of about 120 μm, and the first conductor 113 is disposed around the second pores at a diameter of about 270 μm. The interval between the second pores is about 600 μm.

A second electrode layer 110′₂ has a substantially circular substrate 111, and has a plurality of pores 112, a first conductor 113, a second conductor 114, and an output terminal 115 on the substrate 111.

In this example, the substrate 111 has a diameter of about 5,000 μm, and has an extension section for placing the second conductor 114 on the output terminal 115 side. The extension section has a dimension of about 1,000 μm.

The plurality of pores 112 have first pores having a first diameter and second pores having a smaller diameter than the first diameter. The first pores have a diameter of about 500 μm, and the first conduct 113 is disposed around the first pores at a diameter of about 800 μm. The interval between the first pores is about 1,200 μm. The second pores have a diameter of about 120 μm, and the first conductor 113 is disposed around the second pores at a diameter of about 270 μm. The interval between the second pores is about 300 μm.

The first electrode layer 110′₁ and second electrode layer 110′₂ are configured so that, when overlaid on top and bottom, a central axis of each of the plurality of pores in the first electrode layer 110′ is offset from a central axis of each of the plurality of pores in the second electrode layer 110′₂. This is preferable in that randomization of routes that a plurality of lead wires can take can be promoted as described above.

The first electrode layer 110′₁ and the second electrode layer 110′₂ secure an area where the conductor 113 and/or conductor 114 can be disposed on the substrate 111 while increasing the probability of a plurality of lead wires to pass through a pore by having a plurality of pores with different diameters.

The electrode layers shown in FIG. 4 are suitable for an electrode structure for obtaining electrical signals from a plurality of nerves of an organism. An electrode structure can be formed by disposing the electrode layers shown in FIG. 4 apart from each other. In such an electrode structure, electrode layers having substantially the same pore arrangement as the first electrode layer 110′₁ and electrode layers having substantially the same pore arrangement as the second electrode layer 110′₂ can be, for example, alternatingly disposed. For example, the second electrode layer 110′₂ can be disposed adjacent to the first electrode layer 110′ and a third electrode layer having substantially the same pore arrangement as the first electrode layer 110′, can be disposed adjacent to the second electrode layer 110′₂, whereby the second electrode layer would be disposed between the first electrode layer and the third electrode layer. Furthermore, a fourth electrode layer having substantially the same pore arrangement as the second electrode layer 110′₂ can also be disposed adjacent to the third electrode layer, whereby the third electrode layer would be disposed between the second electrode layer and the fourth electrode layer. In any adjacent electrode layers in such an electrode structure, a central axis of each of the plurality of pores in one of the electrode layers would be offset from a central axis of each of the plurality of pores in the other electrode layer.

When an electrical signal is obtained from a plurality of lead wires by using the electrode structure 100, the plurality of lead wires are in a state where the wires have passed through the electrode structure 100, as described above. If the plurality of lead wires are a plurality of members with electrical conductivity such as a copper wire, wire, or cable, the plurality of members can be, for example, randomly passed through a pore of each electrode layer manually. If the plurality of lead wires are a plurality of nerves of an organism, the plurality of nerves can be, for example, allowed to randomly pass through a pore of each electrode layer via a growth process of the nerves.

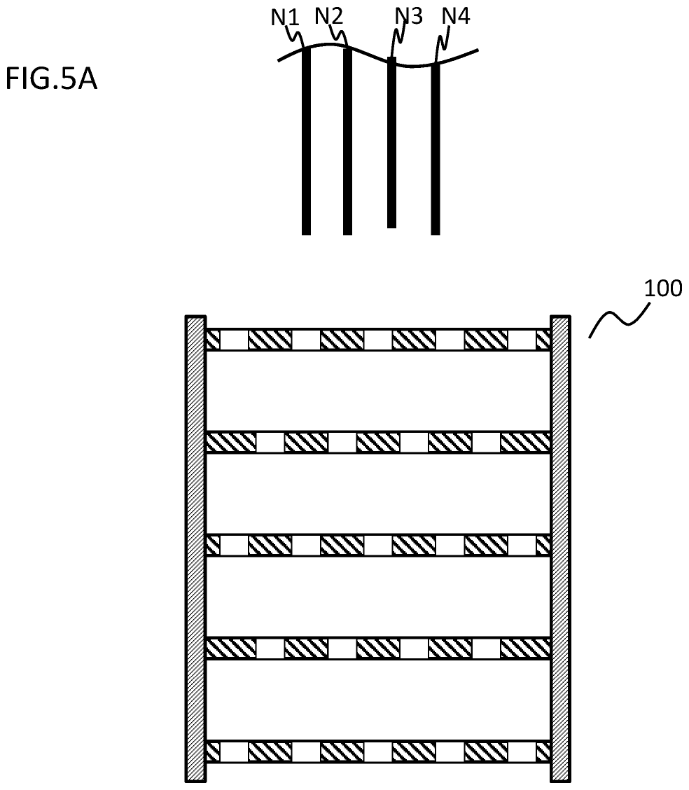

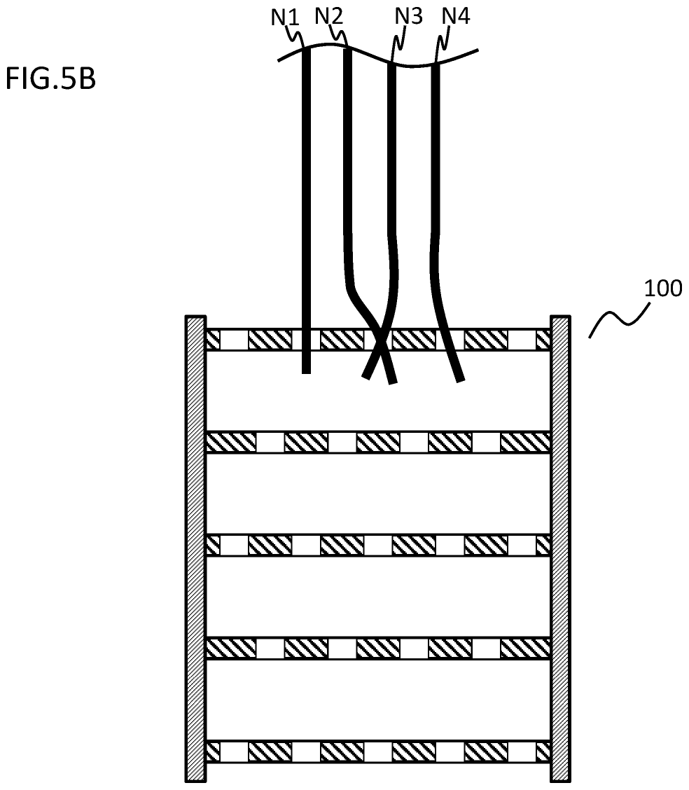

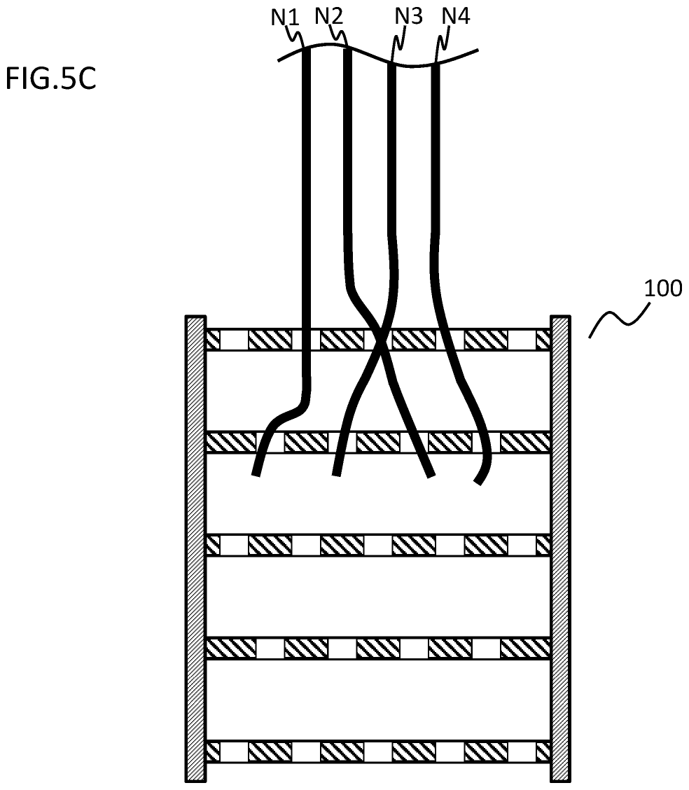

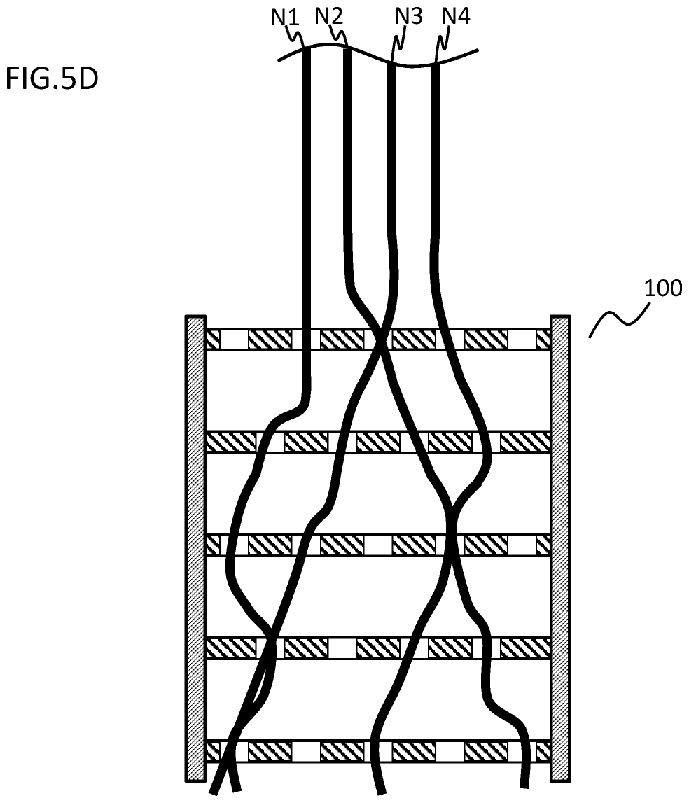

FIGS. 5A to 5D show an example of a plurality of nerves growing in the electrode structure 100 according to one embodiment of the invention.

FIGS. 5A to 5D are described using four nerves N1 to N4 to simplify the description. The number of plurality of nerves is not limited thereto. Any number of nerves can be used with the electrode structure 100.

FIGS. 5A to 5D show the cross-section of the electrode layer 100. In this example, the electrode structure 100 has five electrode layers. In the following descriptions, the electrode layers are referred to as the first electrode layer, second electrode layer, third electrode layer, fourth electrode layer, and fifth electrode layer, in order from the top. Each electrode layer has a plurality of pores. In the following descriptions, pores are referred to as the first pore, second pore, third pore . . . in order from the left in each electrode layer. The m-th pore of the n-th electrode layer is referred to as poreₘₘ. The first electrode layer, third electrode layer, and electrode fifth layer have substantially the same electrode arrangement, and the second electrode layer and the fourth electrode layer have substantially the same electrode arrangement, whereby a central axis of each of the plurality of pores in one of adjacent electrode layers is offset from a central axis of each of the plurality of pores in the other electrode layer.

As shown in FIG. 5A, the four nerves N1 to N4 grow from above the electrode structure 100.

When the four nerves N1 to N4 grow, the nerves pass through a pore of the first electrode layer, as shown in FIG. 5B. For example, pore₁₂ is in the direction of growth (top to bottom direction on the page) of the first nerve N1. While the first nerve N1 can continue to grow in said direction of growth, there is no pore in the direction of growth (top to bottom direction on the page) of the second nerve N2. Thus, the second nerve N2 would grow by changing the direction of growth so that the nerve can pass through a pore. At this time, which direction the direction of growth of the nerve is changed to is determined in accordance with, for example, the behavioral policy of the nerve. Examples of the behavioral policy of a nerve include a policy of following other nerves or structures, etc.

In the example shown in FIG. 5B, the first nerve N1 passes through the second pore₁₂ of the first electrode layer, the second nerve N2 passes through the third pore₁₃ of the first electrode layer, the third nerve N3 passes through the third pore₁₃ of the first electrode layer, and the fourth nerve N4 passes through the fourth pore₁₄ of the first electrode layer.

When the four nerves N1 to N4 grow further, the nerves would pass through a pore of the second electrode layer, as shown in FIG. 5C. Each nerve would grow by changing the direction of growth thereof so that the nerve can pass through a pore.

When the four nerves N1 to N4 grow further, the nerves would pass through pores of the third and fourth electrode layers, as shown in FIG. 5D.

In this manner, the four nerves N1 to N4 can pass through the electrode structure 100. The first nerve N1 has a route passing through pore₁₂-pore₂₁-pore₃₁-pore₄₁-pore₅₁. The second nerve N2 has a route passing through pore₁₃-pore₂₂-pore₃₂-pore₄₁-pore₅₁. The third nerve N3 has a route passing through pore₁₃-pore₂₃-pore₃₄-pore₄₃-pore₅₃. The fourth nerve N4 has a route passing through pore₁₄-pore₂₄-pore₃₄-pore₄₄-pore₅₅. In this manner, the route each nerve follows is unique.

An electrical signal can be obtained from a plurality of nerves in a state where the plurality of nerves have passed through the electrode structure 100, as shown in FIG. 5D. If, for example, an electrical signal is detected from pore₁₂, pore₂₁, pore₃₁, pore₄₁, and pore₅₁, the electrical signals can be identified as an electrical signal that propagates through the first nerve N1 having a route passing through pore₁₂-pore₂₁-pore₃₁-pore₄₁-pore₅₁. If, for example, an electrical signal is detected from pore₁₄, pore₂₄, pore₃₄, pore₄₄, and pore₅₅, the electrical signals can be identified as an electrical signal that propagates through the fourth nerve N4 having a route passing through pore₁₄-pore₂₄-pore₃₄-pore₄₄-pore₅₅.

For example, an electrical signal that propagates through a nerve can be distinguished from noise from the propagation rate of the electrical signal that propagates through a nerve, the distance between electrode layers, the position of a pore at which the electrical signal has been detected, and the time at which the electrical signal has been detected. If the time at which the electrical signal has been detected is significantly earlier or later than the expected time for a nerve having a certain route in view of the propagation rate of the electrical signal that propagates through the nerve, the distance between electrode layers, and the position of a pore, the electrical signal can be determined to be noise. When an electrical signal has been detected from pore₁₃, pore₂₃, pore₃₃, pore₃₄, pore₄₃, and pore₅₃ in one example, the electrical signal detected from pore₃₃ can be determined to be noise if the time at which the electrical signal has been detected at pore₃₃ is significantly earlier or later than the expected time for a nerve passing through pore₁₃ and pore₂₃ in view of the propagation rate of the electrical signal that propagates through a nerve, the distance between electrode layers, and the position of the pore. In addition, the electrical signals that have been detected at pore₁₃, pore₂₃, pore₃₄, pore₄₃, and pore₅₃ can be identified as an electrical signal that propagates through the second nerve N2 having a route passing through pore₁₃-pore₂₃-pore₃₄-pore₄₃-pore₅₃.

For example, a plurality of electrical signals can be distinguished from the propagation rates of the electrical signals that propagate through a nerve, distance between electrode layers, positions of pores at which an electrical signal has been detected, and times at which an electrical signal has been detected. If the time at which an electrical signal has been detected is significantly earlier or later than the expected time for a nerve having a certain route in view of the propagation rate of the electrical signal that propagates through the nerve, the distance between electrode layers, and the position of a pore, the electrical signal can be determined as not originating from the nerve. When an electrical signal has been detected from pore₁₃, pore₁₄, pore₂₃, pore₂₄, pore₃₄, pore₄₃, pore₄₄, pore₅₃, and pore₅₅ in one example, the electrical signal detected from pore₄₄ and the electrical signal detected from pore₅₃ can be determined as having different routes if the time at which an electrical signal has been detected at pore₅₃ is significantly earlier or later than the expected time for a nerve passing through pore₄₄ in view of the propagation rate of the electrical signal that propagates through the nerve, the distance between electrode layers, and the position of a pore. If the time at which an electrical signal has been detected at pore₂₄ is significantly earlier or later than the expected time for a nerve passing through pore₁₃ in this example, the electrical signal that has been detected from pore₂₄ can be determined as having a different route from the electrical signal that has been detected from pore₁₃. In view of the above, as a possible combination of pore₁₃, pore₁₄, pore₂₃, pore₂₄, pore₃₄, pore₄₃, pore₄₄, pore₅₃, and pore₅₅, electrical signals that have been detected from pore₁₃, pore₂₃, pore₃₄, pore₄₃, and pore₅₃ can be identified as an electrical signal that propagates through the second nerve N2 having a route passing through pore₁₃-pore₂₃-pore₃₄-pore₄₃-pore₅₃ and electricals signal that have been detected from pore₁₄, pore₂₄, pore₃₄, pore₄₄, and pore₅₅ can be identified as an electrical signal that propagates through the fourth nerve N4 having a route passing through pore₁₄-pore₂₄-pore₃₄-pore₄₄-pore₅₅. If, for example, there is no route corresponding to a route passing through a pore at which an electrical signal has been detected, the electrical signal that has been detected at the pore can be determined as noise.

If, for example, a plurality of electrical signals cannot be distinguished at a certain time, it is possible to wait for another electrical signal until an electrical signal with a different timing is detected. If an electrical signal with a different timing still cannot be detected, the electrical signals are understood to be from a plurality of redundant nerves. In such a case, the electrical signals do not need to be separated. Specifically, a plurality of electrical signals are distinguished by using the electrode structure 100 for separating the degree of freedom of control of nerves rather than for separating each nerve.

It is preferable that a plurality of electrical signals are distinguished, for example, based on electrical signals detected in multiple samplings rather than based on only an electrical signal detected in one sampling, because there can be a layer or a pore where measurement is not possible due to, for example, an issue in an electrode or an issue in sampling. If the majority of electrical signals are consistent as a whole in multiple samplings even with the presence of a layer or pore where measurement is not possible, the electrical signals can be separated from other electrical signals as effective electrical signals.

If, for example, the electrode structure 100 has a configuration that can receive an electrical signal from the outside and communicate the received electrical signal to a pore within the electrode structure in one embodiment, stimulation can be applied to a nerve passing through a pore. At this time, the route the nerve passes through can be identified by applying stimulation to a pore and detecting propagation of the stimulation at pores of each layer. This enables, for example, route the nerve passes through to be identified, and in turn an electrical signal that propagates through the nerve passing through the route to be distinguished from other electrical signals, even when an electrical signal with a different timing is not detected as described above.

In this manner, a plurality of electrical signals can be obtained from a plurality of lead wires by allowing a plurality of lead wires (e.g., plurality of nerves) to pass through the electrode structure 100.

Obtaining a plurality of electrical signals from a plurality of lead wires can be materialized by, for example, system 1000 described below.

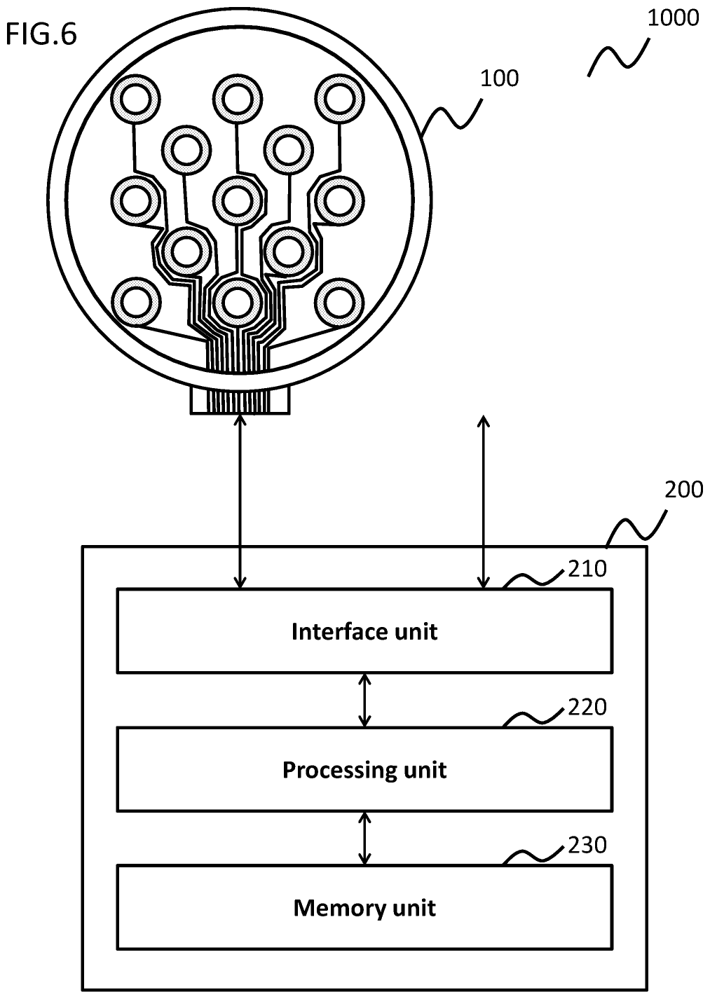

FIG. 6 shows an example of a configuration of the system 1000 for obtaining electrical signals from a plurality of lead wires, according to one embodiment of the invention.

The system 1000 comprises the electrode structure 100 and a controller 200.

The description of the electrode structure 100 is omitted because the electrode structure has the same configuration as the electrode structure 100 describes above. One electrode structure 100 is shown in the example in FIG. 6, but the number of electrode structures 100 the system 1000 comprises is not limited thereto. The system 1000 can comprise at least one electrode structure 100.

If, for example, a plurality of electrode structures 100 are embedded into an organism and used to obtain a plurality of electrical signals from a plurality of nerves of the organism, the plurality of the electrode structures 100 may be embedded into, for example, the same organism or different organisms. If the plurality of electrode structures 100 are embedded into the same organism, the plurality of electrode structures 100 may be embedded into, for example, the same site or different sites.

The controller 200 comprises an interface unit 210, a processing unit 220, and a memory unit 230.

The interface unit 210 exchanges information with an element external to the controller 200. The processing unit 220 of the controller 200 can receive information from an element external to the controller 200 transmit information to an element external to the controller 200, via the interface unit 210. The interface unit 210 can exchange information in any form.

For example, the interface unit 210 can receive an electrical signal from the electrode structure 100. Specifically, the interface unit 210 functions as receiving means for receiving an electrical signal from the electrode structure 100. The interface unit 210 may be configured to amplify a received signal or to receive an amplified signal.

The interface unit 210 can transmit any output based on an electrical signal received from the electrode structure 100 out of the controller 200. An output can be utilized in any application. For example, an output can be utilized for analyzing a signal flowing within a signal line without cutting the signal line. The interface unit 210 can also transmit an electrical signal to the electrode structure 100, whereby the interface unit 210 can establish a two-way communication with the electrode structure 100.

The interface unit 210 can transmit a control signal to an object connected to the controller 200. In this regard, the control signal can be generated in the processing unit 220 based on an electrical signal received from the electrode structure 100.

An object can be, for example, an external instrument or a biological tissue. An external instrument may be, for example, a body assisting apparatus such as a prosthetic arm or a prosthetic leg, a remotely controllable robot, etc. A biological tissue may be a tissue of an organism into which the electrode structure 100 is embedded, or a tissue of an organism that is different from the organism into which the electrode structure 100 is embedded. The same electrode structure as the electrode structure 100 can be embedded into another organism. A biological tissue can be controlled by transmitting a control signal to the biological tissue. For example, a paralyzed leg can be controlled to move by transmitting a control signal based on the intent of ambulation detected by the electrode structure 100 to the paralyzed leg.

The processing unit 220 executes processing of the controller 200 and controls the overall movement of the controller 200. The processing unit 220 reads out a program stored in the memory unit 230 and executes the program, whereby the controller 200 can function as a system for executing desired steps. The processing unit 220 may be implemented by a single processor or a plurality of processors.

The processing unit 220 can be configured to, for example, distinguish a plurality of electrical signals received via the interface unit 210. Specifically, the processing unit 220 can function as distinguishing means for distinguishing a plurality of electrical signals.

The processing unit 220 can, for example, identify which pore of each electrode layer of the electrode structure 100 that one of the plurality of electrical signals has been detected from to identify which of the plurality of lead wires said one of the plurality of electrical signals propagates through, whereby the processing unit 220 can distinguish said one of the plurality of electrical signals from other electrical signals of the plurality of electrical signals.

The processing unit 220 can distinguish a plurality of electrical signals and/or noise based on, for example, the propagation rate of an electrical signal that propagates through a lead wire, distance between electrode layers, position of a pore at which an electrical signal has been detected, and time at which an electrical signal has been detected.

The processing unit 220 can, for example, determine that an electrical signal is not an electrical signal of a route (e.g., electrical signal of another route or noise) if the time at which the electrical signal has been detected is significantly earlier or later than the expected time for a lead wire having a certain route in view of the propagation rate of the electrical signal that propagates through the lead wire, the distance between electrode layers, and the position of a pore at which the electrical signal has been detected (e.g., earlier or later by about 1.1-fold or more, about 1.2-fold or more, about 1.5-fold or more, about 2-fold or more, etc. than the expected time).

If, for example, the processing unit 220 cannot distinguish a plurality of electrical signals at a certain time, the processing unit can wait for another electrical signal until an electrical signal with a different timing is detected to distinguish the plurality of electrical signals by using the electrical signal with a different timing.

It is preferable that a plurality of electrical signals are distinguished, for example, based on electrical signals detected in multiple samplings rather than based on only an electrical signal detected in one sampling, because there can be a layer or a pore where measurement is not possible due to, for example, an issue in an electrode or an issue in sampling. If the majority of electrical signals are consistent as a whole in multiple samplings even with the presence of a layer or pore where measurement is not possible, the electrical signals can be separated from other electrical signals as effective electrical signals by the processing unit 220.

In one exemplary embodiment, the processing unit 220 can transmit an electrical signal to a lead wire passing through a pore via the interface unit 210 and detect the propagation of the electrical signal at a pore of each layer to identify a route through which the lead wire passes. This enables, for example, the route the lead wire passes through, and in turn an electrical signal that propagates through the route, to be distinguished from other electrical signals, even when an electrical signal with a different timing is not detected.

In one exemplary embodiment, the processing unit 220 may be configured to distinguish a plurality of electrical signals by matching a pattern with patterns of signals detected from the entire electrode structure 100. For example, the processing unit 220 can distinguish a plurality of electrical signals by learning an electrical signal detection pattern upon passage through the electrode structure 100 in advance for each of the plurality of electrical signals that propagate through a plurality of lead wires and determining whether an electrical signal detection pattern obtained upon practice matches any of the learned electrical signal detection patterns.

The memory unit 230 stores a program required for the execution of processing of the controller 200, data that is required for the execution of the program, etc. The memory unit 230 may store a program for processing to distinguish a plurality of electrical signals. In this regard, a program is stored in the memory unit 230 in any manner. For example, a program may be pre-installed in the memory unit 230. Alternatively, a program may be installed into the memory unit 230 by download through a network, which can be connected to via the interface unit 210. In such a case, the network can be of any type. The memory unit 230 can be implemented by any storage means.

In the example shown in FIG. 6, each constituent element of the controller 200 is provided within the controller 200, but the present invention is not limited thereto. Any of the constituent elements of the controller 200 can be provided external to the controller 200. If, for example, each of the processing unit 220 and the memory unit 230 is comprised of separate hardware parts, each hardware member may be connected via any network. At this time, the network may be of any time. Each hardware member may be connected via, for example, a LAN, a wireless connection, or a wired connection. The controller 200 is not limited to a specific hardware configuration. The processing unit 220 comprised of an analog circuit instead of a digital circuit is also within the scope of the invention. The configuration of the controller 200 is not limited to those described above, as long as the function thereof can be materialized.

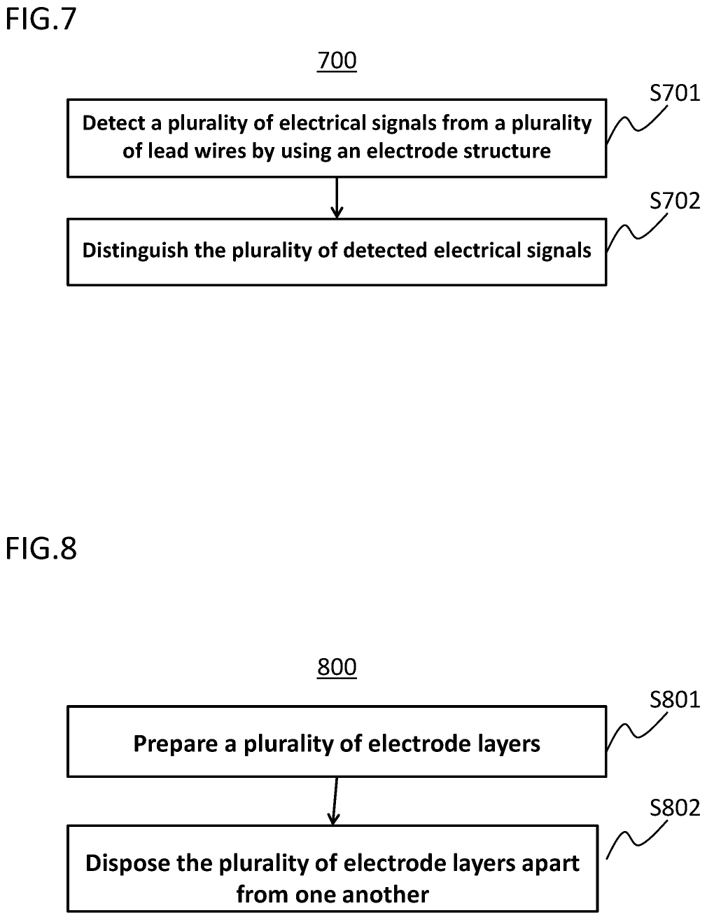

FIG. 7 shows an example of procedure 700 for obtaining electrical signals from a plurality of lead wires. The procedure 700 is performed by using, for example, the system 1000 described above.

At step S701, a plurality of electrical signals are detected from a plurality of lead wires by using the electrode structure 100 of the system 1000.

As described above, the electrode structure 100 comprises a plurality of electrode layers disposed apart from one another, and each of the plurality of electrode layers has a plurality of pores having a dimension that allows passage of a plurality of lead wires. Each of the plurality of pores is connected to a conductor for conducting an electrical signal that propagates through a passing lead wire out from the electrode structure.

Step S701 is performed while a plurality of conductors are passed through the electrode structure 100. At this time, a route the plurality of conductors follows within the electrode structure 100 is a route unique to each of the plurality of conductors. Thus, the electrode structure 100 should have the number of electrode layers and/or number of pores within each electrode layer that can form a significantly greater number of routes than the number of the plurality of conductors in order to allow a unique route for each of the plurality of conductors.

At step S702, the controller 200 of the system 1000 distinguishes the plurality of electrical signals detected at step S701.

At step S702, the interface unit 210 of the controller 200 first receives a plurality of electrical signals from the electrode structure 100. The interface unit 210 can receive a plurality of electrical signals from, for example, the output terminal 115 of the electrode structure 100. The processing unit 220 of the controller 200 then distinguishes the plurality of received electrical signals.

For example, the processing unit 220 can identify which pore of each electrode layer of the electrode structure 100 one of the plurality of electrical signals has been detected from to identify which of the plurality of lead wires said one of the plurality of electrical signals propagates through, whereby the processing unit 220 can distinguish said one of the plurality of electrical signals from other electrical signals of the plurality of electrical signals.

The processing unit 220 can distinguish a plurality of electrical signals and/or noise based on, for example, the propagation rates of the electrical signals that propagate through a lead wire, distance between electrode layers, positions of pores at which an electrical signal has been detected, and time at which an electrical signal has been detected.

The processing unit 220 can, for example, determine that an electrical signal is not an electrical signal of a route (e.g., electrical signal of another route or noise) if the time at which the electrical signal has been detected is significantly earlier or later than the expected time for a lead wire having a certain route in view of the propagation rate of the electrical signal that propagates through the lead wire, the distance between electrode layers, and the position of a pore at which the electrical signal has been detected (e.g., earlier or later by about 1.1-fold or more, about 1.2-fold or more, about 1.5-fold or more, about 2-fold or more, etc. than the expected time).

If, for example, the processing unit 220 cannot distinguish a plurality of electrical signals at a certain time, the processing unit can wait for another electrical signal until an electrical signal with a different timing is detected to distinguish a plurality of electrical signals by using the electrical signal with a different timing.

It is preferable that a plurality of electrical signals are distinguished, for example, based on electrical signals detected in multiple samplings rather than based on only an electrical signal detected in one sampling, because there can be a layer or a pore where measurement is not possible due to, for example, an issue in an electrode or an issue in sampling. If the majority of electrical signals are consistent as a whole in multiple samplings even with the presence of a layer or pore where measurement is not possible, the electrical signals can be separated from other electrical signals as effective electrical signals by the processing unit 220.

In one exemplary embodiment, the processing unit 220 can transmit an electrical signal to a lead wire passing through a pore via the interface unit 210 and detect the propagation of the electrical signal at a pore of each layer to identify a route through which the lead wire passes. This enables, for example, the route the lead wire passes through to be identified, and in turn an electrical signal that propagates through the route to be distinguished from other electrical signals, even when an electrical signal with a different timing is not detected.

In one exemplary embodiment, the processing unit 220 may be configured to distinguish a plurality of electrical signals by matching a pattern with patterns of signals detected from the entire electrode structure 100. For example, the processing unit 220 can distinguish a plurality of electrical signals by learning an electrical signal detection pattern upon passage through the electrode structure 100 in advance for each of the plurality of electrical signals that propagate through a plurality of lead wires and determining whether an electrical signal detection pattern obtained upon practice matches any of the learned electrical signal detection patterns.

A plurality of electrical signals can be obtained in a distinguishable form from a plurality of conductors by the procedure 700. The obtained electrical signals can be utilized in any application. For example, a control signal for controlling an object (e.g., external instrument or biological tissue) can be generated based on an electrical signal. Since a plurality of conductors can also be distinguished, a predefined electrical signal can be conducted to a desired conductor. This enables, for example, transmission of a predefined electrical signal to a desired nerve (e.g., applying electrical stimulation, for controlling to move a paralyzed leg, to a nerve responsible for leg movement).

FIG. 8 shows an example of procedure 800 for manufacturing the electrical structure 100 for obtaining electrical signals from a plurality of lead wires.

At step S801, a plurality of electrode layers 110 are prepared.

Each of the plurality of electrode layers 110 has a plurality of pores having a dimension that allows passage of a plurality of lead wires, as described above. Each of the plurality of pores is connected to a conductor for conducting an electrical signal that propagates through a passing lead wire out from the electrode structure.

The plurality of electrode layers 110 can be manufactured, for example, by etching, in the same manner as generic electronic substrates. The electrode layer 110 can be made by sandwiching the layers with a polyimide film, etc. in the same manner as a flexible substrate, plating an exposed electrode section as needed, etc.

At step S802, the plurality of electrode layers 110 prepared in step S801 are disposed apart from one another.

The plurality of electrode layers 110 are, for example, disposed apart from one another and connected together with the connection member 120 to form the electrode structure 100.

For example, the plurality of electrode layers 110 can be integrally formed to form the electrode structure 100. In such a case, step S801 and step S802 can be performed simultaneously.

The electrode structure 100 is manufactured by the procedure 800. The electrode structure 100 and the system 1000 comprising the electrode structure 100 can be utilized in any application as described above. The electrode structure 100 and the system 1000 comprising the electrode structure 100 can be utilized in, for example, BMI.

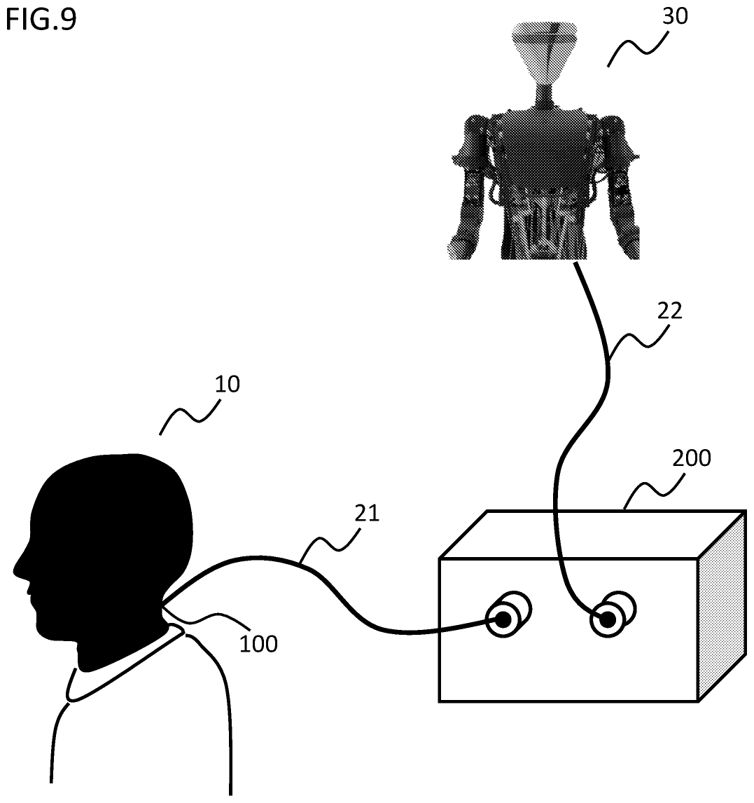

FIG. 9 schematically shows an example of the system 1000 for obtaining electrical signals from a plurality of lead wires according to one embodiment of the invention when used in BMI.

This example describes an example of driving a robot 30 by using an electrical signal obtained directly from the brain of a subject 10 by the electrode structure 100.

The electrode structure 100 is embedded into the body of the subject 10. For example, the electrode structure 100 can be embedded into the spinal cord of the subject 10 or a nerve extending therefrom to the periphery. After the electrode structure 100 is embedded, a nerve extending from the brain is grown into the electrode structure 100, whereby a signal that propagates in a nerve from the brain via the electrode structure 100 propagates into the electrode structure 100.

The electrode structure 100 is connected to the controller 200 via a cable 21. The controller 200 is connected to the robot 30 via a cable 22. In the example shown in FIG. 9, the connection between the electrode structure and the controller 200 and the connection between the controller 200 and the robot 30 are shown as a wired connection, but a wireless connection can be used in place of the wired connection.

### (Learning Stage)

The subject 10 imagines operating the robot 30. For example, the subject imagines causing the robot 30 to walk, causing the robot 30 to grasp an object, causing the robot 30 to drop the grasped object, etc. A corresponding electrical signal is emitted from the brain of the subject 10 by the subject 10 imagining operating the robot 30.

An electrical signal emitted from the brain of the subject 10 when the subject 10 imagines each operation of the robot 30 is obtained via the electrode structure 100. The obtained electrical signal is inputted into the controller 200 via the cable 21. The subject 10 can input what operation was imagined into the controller 200, whereby each operation intended by the subject is associated with the emitted signal and learned.

### (Operation Stage)

The subject 10 imagines operating the robot 30. For example, the subject imagines causing the robot 30 to walk, causing the robot 30 to grasp an object, causing the robot 30 to drop the grasped object, etc. A corresponding electrical signal is emitted from the brain of the subject 10 by the subject 10 imagining operating the robot 30.

An electrical signal emitted from the brain of the subject 10 when the subject 10 imagines each operation of the robot 30 is obtained via the electrode structure 100. The obtained electrical signal is inputted into the controller 200 via the cable 21. The controller 200 determines what operation was intended by the input electrical signal, when the electrical signal is emitted, by referring to the learned information, whereby the controller 200 recognizes the intent of the subject 10 from the obtained electrical signal and transmits a control signal to the robot 30 so that the robot would move as intended.

The robot 30 moves in accordance with the received control signal.

In this manner, the subject 10 can operate the robot 30 simply by imaging operating the robot 30. The robot 30 can be moved as if the robot is the subject 10's own body.

The application in BMI described above is one example. The system 1000 for obtaining electrical signals from a plurality of lead wires can also be utilized in other applications.

The present invention is not limited to the embodiments described above. It is understood that the scope of the present invention should be interpreted based solely on the claims. It is understood that an equivalent scope can be practiced based on the descriptions of the present invention and common general knowledge from the specific descriptions in the preferred embodiments of the invention.

### INDUSTRIAL APPLICABILITY

The present invention is useful for providing a method for obtaining plurality of electrical signals that propagate through a plurality of lead wires in a distinguishable form, etc.

### REFERENCE SIGNS LIST

100 electrode structure
110 electrode layer
111 substrate
112 pore
113, 114 conductor
115 output terminal
120 connection section
200 controller
1000 system

1. A method for obtaining electrical signals from a plurality of lead wires, comprising:
detecting a plurality of electrical signals from the plurality of lead wires by using an electrode structure, the electrode structure comprising a plurality of electrode layers that are disposed apart from one another, each of the plurality of electrode layers having a plurality of pores having a dimension that allows passage of the plurality of lead wires, and each of the plurality of pores being connected to a conductor for conducting an electrical signal that propagates through a passing lead wire out from the electrode structure; and
distinguishing the plurality of detected electrical signals.

2. The method of claim 1, wherein the distinguishing comprises distinguishing the plurality of electrical signals based on a propagation rate of an electrical signal, a distance between the plurality of electrode layers, and a position of a pore and a time at which an electrical signal has been detected.

3. The method of claim 1, wherein the plurality of pores have a first pore having a first dimension, and a second pore having a second dimension, which is different from the first dimension.

4. The method of claim 1, wherein
the plurality of electrode layers comprise at least a first electrode layer and a second electrode layer disposed adjacent to the first electrode layer, and
a central axis of at least one of the plurality of pores in the first electrode layer is offset from a central axis of at least one of the plurality of pores in the second electrode layer.

5. The method of claim 4, wherein
the plurality of electrode layers comprise at least a first electrode layer and a second electrode layer disposed adjacent to the first electrode layer, and
a central axis of each of the plurality of pores in the first electrode layer is offset from a central axis of each of the plurality of pores in the second electrode layer.

6. The method of claim 4, wherein the plurality of electrode layers further comprise a third electrode layer disposed adjacent to the second electrode layer, the second electrode layer is disposed between the first electrode layer and the third electrode layer, and the third electrode layer has substantially the same pore arrangement as the first electrode layer.

7. The method of claim 6, wherein the plurality of electrode layers further comprise a fourth electrode layer disposed adjacent to the third electrode layer, the third electrode layer is disposed between the second electrode layer and the fourth electrode layer, and the fourth electrode layer has substantially the same pore arrangement as the second electrode layer.

8. The method of claim 1, wherein the number of the plurality of electrode layers is 3 or greater, or the number of the plurality of pores is 7 or greater.

9. The method of claim 1, wherein the plurality of lead wires are a plurality of nerves of an organism.

10. An electrode structure for obtaining electrical signals from a plurality of lead wires, the electrode structure comprising a plurality of electrode layers that are disposed apart from one another, each of the plurality of electrode layers having a plurality of pores having a dimension that allows passage of the plurality of lead wires, and each of the plurality of pores being connected to a conductor for conducting an electrical signal that propagates through a passing lead wire out from the electrode structure.

11. A system for obtaining electrical signals from a plurality of lead wires, comprising:
the electrode structure of claim 10;
receiving means for receiving a plurality of electrical signals from the electrode structure; and
distinguishing means for distinguishing the plurality of electrical signals.

12. The system of claim 11, wherein the distinguishing means is configured to distinguish the plurality of electrical signals based on propagation rates of the electrical signals, a distance between the plurality of electrode layers, and positions of pores and times at which an electrical signal has been detected.

13. The system of claim 11 for use in a brain-machine interface (BMI).

14. A method of manufacturing an electrode structure for obtaining electrical signals from a plurality of lead wires, comprising the steps of:
preparing a plurality of electrode layers, each of the plurality of electrode layers having a plurality of pores having a dimension that allows passage of the plurality of lead wires, and each of the plurality of pores being connected to a conductor for conducting an electrical signal that propagates through a passing lead wire out from the electrode structure; and
disposing the plurality of electrode layers apart from one another.

15. The method of manufacturing of claim 14, wherein
the plurality of lead wires are a plurality of nerves of an organism, and
the step of preparing a plurality of electrode layers comprises a step of determining the number of the plurality of electrode layers and the number of the plurality of pores so that the number of trajectories the nerves can take during a process of growth of the nerves is greater than the number of the plurality of nerves.
